(19) United States
(12) Patent Application Publication
(10) Pub. No.: US 2018/0110409 A1
(43) Pub. Date: Apr. 26, 2018

(54) VISUAL FIELD TEST METHOD/PERIMETER USING VIRTUAL REALITY GLASSES/HEADSET AND A SMARTPHONE OR TABLET OR OTHER PORTABLE DEVICE

(71) Applicant: STYLIANOS GEORGIOS TSAPAKIS, ATHENS (GR)

(72) Inventor: STYLIANOS GEORGIOS TSAPAKIS, ATHENS (GR)

(21) Appl. No.: 15/729,003

(22) Filed: Oct. 10, 2017

Related U.S. Application Data
(60) Provisional application No. 62/410,801, filed on Oct. 20, 2016.

(51) Int. Cl. A61B 3/024; G06F 1/16; A61B 3/00

(52) U.S. Cl. CPC A61B 3/024; A61B 3/005; G06F 1/1686

(57) ABSTRACT

Virtual reality glasses are normally used for entertainment (video, movies, games, etc). In this patent they are re-purposed and used for visual field testing.

A method and apparatus are described about a visual field testing method—virtual reality perimeter built with: 1) “virtual reality glasses” using a smartphone or tablet or other portable device (removable device, with CPU inside) as a display, 2) a computer 3) printer, 4) mouse, 5) a second mouse (optional) and 6) specific software.

For the purpose of visual field testing the “virtual reality perimeter” is classified as a Class I medical device (FDA Regulation. 886.1605).

This application is a non-provisional of, and claims the priority of U.S. Provisional patent application No. 62/410,801, filed Oct. 20, 2016, the entire disclosure of which is herein incorporated by reference.

### BACKGROUND

This invention relates to visual field testing and perimeters.

The field of vision is the area in which objects are visible at the same moment during steady fixation of gaze in one direction.

Automated perimetry is a useful method for the assessment of visual fields in many ophthalmic and neurological diseases. The majority of computerized perimeters are specialized pieces of hardware/software. They typically consist of a projection area, an embedded microcontroller, an input device for the operator, and a button for the patient. The patient stares at a central fixation point in a large, white bowl. Lights are flashed at different locations on the display, within a given region of the visual field and the patient has to press a button each time he/she sees a light stimulus, until the threshold, or the stimulus intensity seen 50% of the time, is recorded at each test location. The light stimuli are bright or dim at different stages of the test while some of the flashes are purely to check that the patient is concentrating. Fixation losses occur when the patient reports seeing a stimulus that is presented in the predicted area of the physiologic blind spot. False positives responses occur when a patient presses the button when no stimulus is presented. False negatives responses occur when a patient fails to see a significantly brighter stimulus at a location than was previously seen.

Current perimeters are accurate instruments but they have a number of drawbacks/disadvantages. Visual field testing is a time consuming process. Testing is inconvenient or stressful for ill or elderly patients and people often become anxious or tired keeping their heads still, in the perimeter, throughout the test. There might be a feeling of claustrophobia. The patch covering the eye if often uncomfortable or irritating. The majority of computerized perimeters are specialized pieces of hardware/software. They typically consist of a projection area, an embedded microcontroller, an input device for the operator, and a button for the patient. These machines are built for physician's offices or hospitals and are bulky, heavy and expensive. They are not portable and they cannot be used at the bedside.

The possibility of using virtual reality glasses, for visual field testing, is interesting because they are portable and inexpensive.

There are 2 fundamentally different categories of “virtual reality glasses” with 2 different underlying technologies, as far as the display is concerned. The first/older type has the display built in (usually LCDs in older models). This category works as an “external head mounted monitor” and may include a graphics processing unit (GPU), but it has no CPU (central processing unit), so it needs to be connected to a computer, in order to function. The display is not removable and doesn't work if removed (it is a “passive device”). Connecting the external head mounted monitor (“virtual reality glasses”) to the computer was as simple as plugging in a RGB wire to the graphics card output.

The second/newer category of virtual reality glasses may use a smart-phone or tablet or other portable device as display. They are normally used for entertainment (video, movies, games, etc). The portable device (smart-phone, tablet, etc) includes a CPU (central processing unit), so it has processing capabilities on its own (usually running on Android or iOS or other operating system) and can work autonomously, without being connected to a computer. The portable device is removable. If removed it works on its own. A smart-phone for example works as a mobile phone (it is an “active device”). This second category of virtual reality glasses is different from the first one; it is not a drop-in replacement.

### DESCRIPTION OF THE PRIOR ART

There have been efforts in the past, to use virtual reality glasses (of the first category, with built in displays) for visual field testing.

U.S. Pat. No. 5,737,060 (Filing date: Aug. 16, 1996) “Visual field perimetry using virtual reality glasses”. Abstract “Visual field perimetry may be performed using virtual reality glasses, a computer, a printer and an external mouse.”Claim 2. “The visual field perimeter of claim 1 wherein said virtual reality glasses contain separate displays for each eye and said discrete targets may be rendered independently to each of the said displays.”. In that patent, a computer was required and the first/older category of virtual reality glasses (LCD displays) were used.

At that time (1996), virtual reality glasses of the second category had not been invented yet. Smart-phones/portable devices, as we know them today, had not been invented either. Android and iOS operating systems for smartphones didn't exist. The first iPhone was released on Jan. 9, 2007 while Android version 1.0 was released on Sep. 23, 2008.

U.S. Pat. No. 5,864,384 A (Filing date: Jul. 31, 1996) “Visual field testing method and apparatus using virtual reality ”. Claim 46. “An apparatus as recited in claim 1, wherein said electronic imaging system is a liquid crystal display panel.”The virtual reality glasses used at that patent, had built in LCD displays. The same considerations as before apply. Connecting the external head mounted monitor “virtual reality glasses” to the computer was simple.

At that time portable devices (smart-phones, tablets, etc), couldn't be used for visual field testing because simply they didn't exist. Neither could virtual reality glasses be used, for such portable devices as they didn't exist either.

The recent article: “Testing of Visual Field with Virtual Reality Goggles in Manual and Visual Grasp Modes”, BioMed Research International, Volume 2014 (2014), Article ID 206082, 10 pages, http://dx.doi.org/10.1155/2014/206082 refers to a “head-mounted visor” with built in OLED displays. The “visor” requires an external computer to operate. Bioformatix, apparently markets the “visor” under the name VirtualEye® (http://www.bioformatix.com/perimetry.html). The article makes no claims about ‘smart-phones’, ‘tablets’ or any mobile “devices” for visual field testing, neither are these words included in the text.

Visual reality glasses with built in display/s (head mounted monitors), did not perform well. These virtual reality glasses were usually built for gaming. The diagonal of the display was usually small. This required moving the fixation point, which confused older patients during testing; while custom built virtual reality glasses with bigger displays were more expensive and not widely available.

In all previous patents/methods described, virtual reality glasses of the first/older category were used for visual field testing (with built in displays, no CPUs, “passive devices”). The technology has changed since then.

### BRIEF SUMMARY OF THE INVENTION

A method and apparatus are described about a visual field testing method-“virtual reality perimeter” built with: 1) “virtual reality glasses” (of the second/newer category) using a smartphone or tablet or other portable device (removable device, with CPU inside) as a display, 2) a computer 3) printer, 4) mouse, 5) a second mouse (optional) and 6) specific software.

“Virtual reality glasses” using mobiles devices, are normally used for entertainment (video, movies, games, etc . . . ). In this patent they are re-purposed and used for visual field testing. For the purpose of visual field testing the “virtual reality perimeter” is classified as a Class I medical device (FDA Regulation 886.1605).

This invention uses different underlying technology and equipment than in previous patents. The new hardware is not a drop-in replacement for the old one.

Making the “virtual reality perimeter”/apparatus work is not straightforward because the new hardware is not a functioning the same way as the old one. The mobile device has no RGB connection.

The new method has many advantages/improvements. The removable device has its own CPU/processing power (“active device”), which allows for the images to be sent compressed to the mobile device and decompressed by the mobile device in real time. The removable device communicates with the computer. Also smart-phones, tablets and portable devices are in general, widely available, may have bigger displays, they are inexpensive, perform better and can work autonomously.

The term “virtual reality glasses” next in this patent, refers to any headset/glasses/etc of any suitable design/material that is designed as a cradle for a device—a tablet or smartphone or any other portable device, and may serve as a display for the virtual reality experience. The virtual reality glasses may have camera/s to monitor the eye/s.

The term “portable/mobile device” next in this patent, refers to any smart-phone, tablet or any other portable/mobile device with its own central processing unit (CPU) and display, suitable for use with the virtual reality glasses.

### DETAILED DESCRIPTION OF THE INVENTION

A method and apparatus are described about a visual field testing method-virtual reality perimeter built with: 1) “virtual reality glasses” (of the second/newer category) using a smartphone or tablet or other portable device (removable device, with CPU inside) as a display, 2) a computer 3) printer, 4) mouse, 5) a second mouse (optional) and 6) specific software.

Once the virtual reality perimeter is assembled, the computer and the mobile device need to communicate somehow.

The computer should send and receive data to/from the mobile device at high speed during visual field testing. Making it work is more difficult than before because the hardware is different. One cannot possibly plug in a RGB wire to the graphics card output. There is no RGB connection available on the mobile device.

The Media Transfer Protocol (MTP) is an extension to the Picture Transfer Protocol (PTP) communications protocol that allows media files to be transferred atomically to and from portable devices.

After the mobile device is connected to the computer using a USB wire, the computer usually recognizes the device and establishes a Media Transfer Protocol (MTP) connection. This works if the user wants to transfer photos.

The naïve/simple approach is to use the file system to transfer compressed files (data) to/from the mobile device. This does not work reliably at high speed.

By experimentation it was found that the transfer speed was ultra slow and error prone, when many small compressed files (data) were sent/received. MTP is a slow protocol. The actual file system is implemented by the device, and not by the computer's operating system. This also means that file system recovery tools on the computer will be of no use. By design, MTP devices (like PTP devices) are not treated as a traditional removable drive. This means that most programs on the computer will not recognize the MTP device, limiting the user to software from the device manufacturer or other MTP specific programs and making the implementation of the connection software more difficult.

It was found experimentally, that at high transfer speed the files (data) were randomly corrupted and/or interrupted, so this method was abandoned. Errors during visual field testing may have serious consequences for the vision of the patient.

Establishing a connection suitable for visual field testing between the mobile device (smartphone, tablet, etc) and the computer presents some challenges; it doesn't work out of the box. This is neither a simple task nor obvious. Some ingenuity is required.

Again by experimentation it was found that the most reliable approach was to use a TCP/IP protocol implementation over the USB wire, using a client-server architecture and sockets programming. The data were sent/received compressed over the TCP/IP connection to achieve a higher transfer rate. This approach was successful.

For android devices, this requires that the Google USB/ADB driver is installed on the computer and USB debugging is enabled on the mobile device. USB Debugging should only be enabled when needed. Leaving it enabled all the time is kind of a security risk for that this mode grants high-level access to the device.

The mobile device can also be connected to the computer wirelessly. This method is more difficult for the average user to setup and is not as reliable as a wired connection.

### Algorithms For Visual Field Testing

There are many different—well known—standard strategies/algorithms available for visual field testing, reported in bibliography (SITA, T.O.P., FAST THRESHOLD 3 dB steps, 4-2-1, 4-2, etc), with their advantages and disadvantages.

The algorithms that may be used for visual field testing are not within the scope of this patent. They are well documented/available in the bibliography.

### Software

For testing purposes—as a proof of concept—to compare the virtual reality perimeter with the Humphrey perimeter, software implementing a standard fast threshold 3 dB steps algorithm was developed and tested (using virtual reality glasses and a 6″ inch smart-phone), albeit any other algorithm may be used. (3 dB steps mean that the algorithm proceeds in 3 dB steps to find the luminosity threshold).

The virtual reality perimeter is portable and may be used at the bedside or while the patient is lying on his/her right/left side.

The software monitors the device orientation to make sure that the device is positioned correctly inside the virtual reality glasses by the user, so that the left part of the testing window is in front of the left eye of the patient and the right part of the testing window is in front of the right eye of the patient. If the device is positioned incorrectly (rotated)180° inside the virtual reality glasses, the software shows a message and testing stops, until the problem is fixed. (This feature was added after a user—during testing—accidentally inserted the mobile device rotated 180° inside the virtual reality glasses. The visual field test result was rotated 180° as well, making the visual field defects appear mirrored in the results of the test, which might make the interpretation of the results error prone in some cases and have serious consequences for the vision of the patient). This is probably the only “virtual reality perimeter” with this feature. Neither of the previous patents about visual field testing using “virtual reality glasses” has this feature nor are there any reports in the literature.

Brightness adjustment. Brightness sets the black point, which determines the low light output level (black level) of the display. The results of a visual field test depend on the luminosity of the examination display. The luminosity must be fixed and standard; in order to make sure the data are consistent from one visit to another, between successive tests, so that the results can be analyzed over time. This also makes it possible to compare data between different installations. A typical display has luminosity approximately 250 cd/m2. Different smartphones models/portable devices might be used for visual field testing. The software allows the user to adjust the device's display brightness (luminosity) using a grayscale step wedge. It should be set to a point that makes all distinct shades of gray clearly visible. This is probably the only “virtual reality perimeter” with this feature. Neither of the previous patents about visual field testing has this feature nor are there any reports in the literature.

The software was able to detect the blind spot automatically by projecting stimuli at possible blind spot locations and checking the responses of the patient. This can also be done manually by the operator. The software monitored fixation using the virtual reality glasses camera/s and/or by projecting stimuli at the blind spot and checking if the patient responded or not. If the patient didn't respond it was assumed that fixation is maintained (This is the well-known Heijl-Krakau method for fixation monitoring, it is outside of the scope of this patent). Each eye may be tested separately, or both eyes at the same time. The light stimuli may be shades of: a) white on gray/black, b) black on gray/white, or c) yellow on blue at user selection (the type of stimuli is outside of the scope of the patent). At the end of the test, the software computed the statistical indexes: test duration, total stimuli projected, false positive, false negative, short fluctuation (SF), mean sensitivity, mean deviation (MD), pattern deviation (PSD), etc. False positive points are printed in green and false negative points are printed in red. Points with both, false positive and false negative responses are printed in yellow.

Although the descriptions above contain specificities, these should not be construed as adding new matter or limiting the scope of the patent but as merely providing illustrations of some of the presently preferred embodiments of this invention.

The details are provided so that a skilled in the art person could avoid pitfalls and practice the invention without “undue experimentation”.

### Examination Procedure

During testing the patient sits comfortably, puts on the virtual reality glasses and adjusts the head straps. The virtual reality glasses should not be tilted, off center, too high or too low. Interpupillary distance is adjusted with the rotating knob on top. Instead of 2 slightly overlapping pictures, the patient should see one picture. To optimize image quality, focus distance is adjusted with the 2 rotating knobs on the sides (may turn simultaneously—depending on the model of virtual reality glasses). The picture should not be blurry but sharp. The patient is free to change position or move his/her head while testing. Virtual reality glasses are not heavy. The patient may hold the virtual reality glasses using his/her hand to make testing more comfortable. The virtual reality glasses must be positioned appropriately or a typical lens rim artifact might occur. Rim lens artifact does not always form a complete rim around the outer edge of the field, but can be partial, and can mimic nasal steps. According to a study (Lens rim artifact in automated threshold perimetry. Zalta A H. Ophthalmology. 1989 Sep; 96(9):1302-11), in central static threshold visual fields (Humphrey 30-2 Program) performed with a corrective lens, lens rim artifact (LRA) was present in 10.4% of 704 fields examined retrospectively and 6.2% of 276 fields evaluated prospectively. The software may locate the blind spot automatically, and adjust the location and size of the test points. Also the location and size of test points can be set manually. Each eye may be tested separately or both eyes at the same time. No eye patch is used. During testing, the patient stares at the central fixation point, while using a mouse to click whenever he/she sees a visual stimulus on the display, whether bright or dim.

### Tests—Results

15 eyes of 10 patients consecutively presented at visual fields lab were tested successively using a Humphrey perimeter and the virtual reality perimeter as described above, within hours for comparison. The patients tolerated the virtual reality visual field test very well. Almost all of them (9 out of 10) reported that it was much more comfortable compared to the standard perimeter (bowl) test. The results were statistically analyzed and compared. Lens rim artifact occurred in one patient during testing with virtual reality glasses. When lens rim artifact occurred the test was repeated.

Statistical analysis. Point to point correlation coefficient (r) between the virtual reality perimeter and the Humphrey perimeter was computed for each eye and for all eyes together using the Instat version 3.05 of GraphPad Software, Inc. When values distribution was not normal the nonparametric Spearman correlation coefficient (r) was used. Virtual Reality Glasses perimetry tests were 24° (52 points) while Humphrey tests were 30° (76 points). Only the corresponding (common 52 points) between these were taken into consideration.

### Results

| | MEAN | | | |
|---|---|---|---|---|
| EYE | DIFFERENCE | SD | SPEARMAN (r) | P (one-tailed) |
| 1 | −1.86538 | 6.594795 | 0.736955 | P < 0.0001 |
| 2 | −8.53846 | 4.90298 | 0.765154 | P < 0.0001 |
| 3 | −6 | 5.1637 | 0.875855 | P < 0.0001 |
| 4 | −3.96154 | 2.449182 | 0.792082 | P < 0.0001 |
| 5 | −4.15385 | 3.754133 | 0.773847 | P < 0.0001 |
| 6 | −2.71154 | 5.163674 | 0.75502 | P < 0.0001 |
| 7 | −6.57692 | 2.717742 | 0.865649 | P < 0.0001 |
| 8 | −3.59615 | 6.698726 | 0.833976 | P < 0.0001 |
| 9 | −2.26923 | 2.870508 | 0.838132 | P < 0.0001 |
| 10 | −7.44231 | 5.146533 | 0.766863 | P < 0.0001 |
| 11 | −3.23077 | 2.422245 | 0.870688 | P < 0.0001 |
| 12 | −5 | 2.828427 | 0.848471 | P < 0.0001 |
| 13 | −3.01923 | 2.313561 | 0.850762 | P < 0.0001 |
| 14 | −4.84615 | 2.154654 | 0.889794 | P < 0.0001 |
| 15 | −2.26923 | 9.614359 | 0.745111 | P < 0.0001 |

Total Results

| MEAN DIFFERENCE | MEAN SD | SPEARMAN (r) | P (one-tailed) |
|---|---|---|---|
| −4.365 | 4.2593 | 0.8139 | P < 0.0001 |

In each eye and in all eyes together the Spearman (r) correlation coefficient value between the two methods was statistically extremely significant (P<0.0001). The high correlation coefficient (0.8139) between the virtual reality perimeter and the Humphrey perimeter shows that the method is reliable at least when compared to the Humphrey perimeter.

What is claimed is:

1. A method and apparatus for visual field testing/“virtual reality perimeter” —comprising of:
(a) 1) “virtual reality glasses” (of the second/newer category) using a smartphone or tablet or other portable device (removable device, with CPU inside) as a display,
b) computer c) printer, d) mouse, e) a second mouse (optionally) and f) specific software for visual field testing, where the mobile device communicates with the computer.

2. The visual field test method/“virtual reality perimeter” of claim 1 wherein the term “virtual reality glasses” refers to any headset/glasses/cardboard of any suitable design/material, that is designed as a cradle for a device—a tablet or smartphone or any other portable device, that may serve as a display for the virtual reality experience, the virtual reality glasses may have cameras to monitor the eye/s.

3. The visual field test method/“virtual reality perimeter” of claim 1 wherein the term “portable device” refers to any smart-phone, tablet or any other portable/mobile device with its own central processing unit (CPU) and display, suitable for use with the virtual reality glasses.
Specific software for visual field testing according to claim 1, which is able to monitor the device's orientation inside the virtual reality glasses and also allows the user to adjust the display luminosity of the mobile device.
